(19) United States
(12) Patent Application Publication
(10) Pub. No.: US 2013/0190403 A1
(43) Pub. Date: Jul. 25, 2013

(54) ADMINISTRATION OF INTRAVENOUS IBUPROFEN TO TRAUMA PATIENTS

(71) Applicants: Leo Pavliv, Cary, NC (US); Amy Dix Rock, Nashville, TN (US)

(72) Inventors: Leo Pavliv, Cary, NC (US); Amy Dix Rock, Nashville, TN (US)

(73) Assignee: Cumberland Pharmaceuticals Inc., Nashville, TN (US)

(21) Appl. No.: 13/356,121

(22) Filed: Jan. 23, 2012

(51) Int. Cl. A61K 31/192; A61P 25/00; A61P 25/04

(52) U.S. Cl. 514/570

(57) ABSTRACT

A method for treating human patients who require hospitalization for a trauma injury wherein the patients are intravenously administered a first dose of a therapeutically effective dose of ibuprofen intravenously as soon as possible after hospitalization is disclosed. The patients are further treated with intravenous ibuprofen at suitable dosing intervals to the human patient until (a) the patient no longer requires intravenous dosing of ibuprofen.

### FIELD OF THE INVENTION

Provided are methods for treating pain in patients suffering from trauma injuries requiring hospitalization and/or reducing the need for narcotic analgesics and/or reducing the length of hospital stay in such patients by intravenously administering a pharmaceutical composition comprising an effective amount of 2-(4-isobutylphenyl) propionic acid.

### BACKGROUND OF THE INVENTION

2-(4-isobutylphenyl) propionic acid, whose International Nonproprietary Name is ibuprofen, is a well-known anti-inflammatory drug having a molecular weight of 206.28 and the following chemical structure:

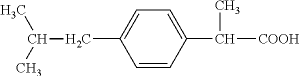

(Merck Index 12th ed., n4925, page 839). Originally patented in the 1960's, ibuprofen is now marketed generically, as well as under the trade names of Motrin®, Advil®, and Nuprin® for the treatment of pain, inflammation, and fever. The U.S. Food and Drug Administration recently approved a new formulation of ibuprofen for intravenous administration to be marketed under the trade name Caldolor®.

Ibuprofen is readily available as the racemic mixture ((RS)-Ibuprofen) of the two enantiomers, (R)-Ibuprofen and (S)-Ibuprofen. Even though the (S) enantiomer is the biologically active form, most preparations contain the racemic mixture since the (R) enantiomer is converted to the active (S) form in-vivo. For simplicity, hereinafter the term “ibuprofen” will be used to indicate any one of the (R) enantiomer, the (S) enantiomer, or the racemate.

Ibuprofen is currently approved for use as oral treatment for minimal to moderate pain from arthritis, surgery, sunburn, menstruation, and fever. Like aspirin and other drugs in the NSAID family, ibuprofen is believed to reduce the inflammatory response by inhibiting the formation of prostaglandins. Several studies have demonstrated the success of oral or rectal ibuprofen in the reduction of fever and the subjective symptoms associated with it.

Although ibuprofen has many advantages over other analgesics such as aspirin and acetaminophen, it is very poorly soluble in water. Thus, certain dosage forms of ibuprofen, especially injectable liquids, have been difficult to develop. Several U.S. patents have addressed this problem.

For example, U.S. Pat. No. 4,309,421 appears to describe water-soluble complexes of ibuprofen and phospholipids suitable for parenteral administration. U.S. Pat. Nos. 4,859,704 and 4,861,797 appear to describe the synthesis of alkali metal salts of ibuprofen for preparing a liquid ibuprofen formulation.

Other U.S. patents appear to address this problem by preparing an ibuprofen salt with a basic amino acid as the active pharmaceutical ingredient and then solubilizing the salt to produce a liquid dosage form.

For example, U.S. Pat. No. 5,200,558 appears to describe enhanced analgesic effects of S (+) ibuprofen as salts of L and D amino acids, including arginine, in various dosage forms, including as an injectable solution. U.S. Pat. No. 4,279,926 appears to describe the use of basic amino acid salts of propionic acids for relieving pain and treating inflammatory conditions. Similarly, U.S. Pat. No. 5,463,117 appears to describe the preparation of salts of ibuprofen with basic amino acids. Finally, U.S. Pat. No. 6,005,005 appears to describe a liquid composition for oral use containing ibuprofen and arginine.

U.S. Pat. No. 6,727,286 B2 describes, among other things, a pharmaceutical composition comprising an aqueous solution of arginine and ibuprofen, wherein the molar ratio of arginine to ibuprofen is less than 1:1, as well as a method of making the same. That patent also provides a method of treating a condition chosen from pain, inflammation, fever, and/or other conditions alleviated by ibuprofen comprising administering a pharmaceutical composition comprising an aqueous solution of arginine and ibuprofen, wherein the molar ratio of arginine to ibuprofen is less than 1:1. The entire contents of U.S. Pat. No. 6,727,286 B2 are hereby incorporated herein by reference.

The U.S. Food and Drug Administration recently approved a new formulation of ibuprofen for intravenous administration to be marketed under the trade name Caldolor® by Cumberland Pharmaceuticals, Inc. Caldolor® contains the active ingredient ibuprofen. As described on the labeling for Caldolor®, “each 1 mL of solution contains 100 mg of ibuprofen in Water for Injection, USP. The product also contains 78 mg/mL arginine at a molar ratio of 0.92:1 arginine:ibuprofen. The solution pH is about 7.4.” Caldolor® is sterile and is intended for intravenous administration only.

Caldolor® possesses antiinflammatory, analgesic, and antipyretic activity. As such, Caldolor® is indicated in adults for the management of mild to moderate pain and the management of moderate to severe pain as an adjunct to opioid analgesics. 400 mg to 800 mg of Caldolor® is administered intravenously every 6 hours as necessary to treat pain. Caldolor® is also indicated for the reduction of fever in adults. 400 mg of Caldolor® is administered intravenously, followed by 400 mg every 4 to 6 hours or 100-200 mg every 4 hours as necessary to treat fever.

Physicians have options in terms of pain and fever control, but each seems to come with a trade-off. It would be highly desirable to provide a new development in the management of pain and fever which improves patient care.

Prior publications report that analgesic potency may be improved while reducing undesirable effects by combining an opioid with an NSAID or an analgesic such as acetylsalicylic acid or acetaminophen, in such a way as to obtain a synergistic analgesic effect allowing for a reduction in the total dose of both the NSAID and analgesic. For example, U.S. Pat. No. 4,569,937, issued to Baker et al. on Feb. 11, 1986, describes a combination of oxycodone with ibuprofen in a ratio of oxycodone/ibuprofen from 1:6 to about 1:400. U.S. Pat. No. 4,690,927, issued to Voss et al. on Sep. 1, 1987, describes a combination of the NSAID diclofenac and codeine in a weight ratio of diclofenac to codeine of about 1:1 to about 3:1. U.S. Pat. No. 5,190,947, issued to Riess et al. on Mar. 2, 1993, describes a diclofenac-codeine salt ([2-[2,6-dichlorophenyl)-amino]-phenyl]-acetic acid). U.S. Pat. No. 4,844,907, issued to Elger et al. on Jul. 4, 1989, describes a multiphase tablet combining a narcotic analgesic phase and an NSAID phase in separate layers. U.S. Pat. No. 4,587,252, issued to Arnold et al. on May 6, 1986, describes a process for treating pain using a combination of hydrocodone and ibuprofen.

### OBJECTS AND SUMMARY OF THE INVENTION

It is an object of the present invention to provide a method for the treatment of pain in human patients who have experienced a trauma injury which requires hospitalization.

It is an object of the present invention to provide a method for the treatment of pain in human patients who have experienced a trauma injury which requires hospitalization which reduces their need for opioid (narcotic) analgesics.

It is another object of the invention to provide a treatment for pain which has favorable risk/benefit safety profile.

It is another object of the invention to provide a method of treating human patients who have experienced a trauma injury which requires hospitalization in a manner which reduces opioid side effects.

It is another object of the invention to provide a method of treating human patients who have experienced a trauma injury which requires hospitalization to be treated with opioid analgesics in a manner which is opioid sparing.

It is another object of the invention to provide a method of treating patients who have experienced an injury requiring hospitalization.

It is another object of the invention to provide a method of treating pain in patients who are being admitted to a hospital for a painful injury.

It is another object of the invention to reduce the length of stay for patients admitted to a hospital for a painful injury.

In accordance with the above objects and others, the present invention is directed in part to the administration of intravenous ibuprofen to human patients who have experienced a trauma injury which requires hospitalization. In certain embodiments, the invention is directed to a method for treating human patient, comprising hospitalizing a human patient who has experienced a trauma injury which requires hospitalization; intravenously administering a therapeutically effective dose of ibuprofen intravenously on the first day of hospitalization; and thereafter intravenously administering a therapeutically effective dose of ibuprofen at suitable dosing intervals to the human patient until the patient no longer requires intravenous dosing of ibuprofen or is discharged from hospitalization.

In certain embodiments, the invention is directed to a method for treating human patient, comprising hospitalizing a human patient who has experienced a trauma injury which requires hospitalization; intravenously administering an therapeutically effective dose of ibuprofen intravenously as soon as possible after hospitalization; and thereafter intravenously administering a therapeutically effective dose of ibuprofen at suitable dosing intervals to the human patient until the patient no longer requires intravenous dosing of ibuprofen or is discharged from hospitalization. In certain preferred embodiments, a suitable therapeutically effective dose of ibuprofen is administered intravenously at suitable dosing intervals for at least 48 hours following hospital admission, before conversion to oral ibuprofen.

In certain preferred embodiments, the dose of ibuprofen is from about 400 mg to about 800 mg.

In certain preferred embodiments, the dosing interval is from about 4 to about 6 hours.

In certain embodiments, the method further comprises administering to the human patient a therapeutically effective dose of an opioid analgesic, and thereafter administering a therapeutically effective dose of an opioid analgesic in suitable dosing intervals to the human patient until the patient no longer requires treatment with an opioid analgesic, or is discharged from hospitalization.

In certain embodiments, the human patient(s) also receiving a therapeutically effective dose of an opioid analgesic is being administered a dose of opioid analgesic that is reduced as compared to a human patient(s) who is hospitalized and being treated for a similar injury who is not receiving concomitant therapy with intravenous ibuprofen. Thus, in certain embodiments, the invention is directed in part to the administration of intravenous ibuprofen to patients who have experienced a trauma injury which requires hospitalization who are being treated with an opioid analgesic(s) for pain, and thereby providing an opioid-sparing effect, enabling the reduction of the dose of opioid to the patient. In certain preferred embodiments, a suitable therapeutically effective dose of ibuprofen is administered intravenously at suitable dosing intervals for at least 6 hours, for at least about 12 hours, for at least about 24 hours, for at least about 36 hours, or for at least about 48 hours following hospital admission, before conversion to oral ibuprofen.

In certain embodiments, the administration of intravenous ibuprofen as detailed above may provide a reduction in side effects associated with the administration of opioid analgesics.

Further, in certain embodiments, the administration of intravenous ibuprofen as detailed above may reduce pain scores (e.g., VAS scores) in patients who are concurrently administered opioid analgesics, as compared to similar patients receiving opioid analgesics alone.

In certain preferred embodiments, the method further comprises reducing the length of stay (i.e., length of hospitalization) via the administration of intravenous ibuprofen as detailed above.

In further embodiments, the administration of intravenous ibuprofen allows the patient to become ambulatory at an earlier time point than if the intravenous ibuprofen is not administered.

In further embodiments, the invention is directed in part to a method of treating human patients who have experienced a rib fracture injury which requires hospitalization for pain, comprising administering an effective dose of intravenous ibuprofen on the first day of hospitalization, and continuing intravenous administration of an effective dose of ibuprofen at suitable dosing intervals to the human patient until the patient no longer requires intravenous dosing of ibuprofen or is discharged from hospitalization. In certain preferred embodiments, the dosing interval is from about every 4 to about every 6 hours after the administration of the first dose of intravenous ibuprofen. In certain preferred embodiments, a suitable therapeutically effective dose of ibuprofen is administered intravenously at suitable dosing intervals for at least 6 hours, for at least about 12 hours, for at least about 24 hours, for at least about 36 hours, or for at least 48 hours following hospital admission, before conversion to oral ibuprofen.

In certain preferred embodiments, the methods of the invention utilize a dose of about 800 mg intravenous ibuprofen administered every 6 hours starting with the first day of hospitalization.

In certain embodiments, the trauma injury does not require surgery.

In additional embodiments, the invention is directed to a method for treating a human patient(s), comprising hospitalizing a human patient who has experienced a trauma injury which requires hospitalization; intravenously administering a therapeutically effective dose (e.g., about 800 mg) of ibuprofen intravenously as soon as possible after hospitalization; and thereafter intravenously administering a therapeutically effective dose of ibuprofen at suitable dosing intervals to the human patient; performing surgery on the patient; and thereafter continuing the intravenous administration of ibuprofen at suitable dosing intervals until the patient no longer requires intravenous dosing of ibuprofen or is discharged from the hospital. In certain preferred embodiments, a suitable therapeutically effective dose of ibuprofen is administered intravenously at suitable dosing intervals for at least 6 hours, for at least about 12 hours, for at least about 24 hours, for at least about 36 hours, or for at least 48 hours following hospital admission, before conversion to oral ibuprofen.

In further embodiments, the invention is directed to a method for treating human patients suffering from a rib fracture, comprising hospitalizing a human patient who has experienced a rib fracture; intravenously administering a therapeutically effective dose (e.g., about 800 mg) of ibuprofen intravenously as soon as possible after hospitalization; thereafter intravenously administering a therapeutically effective dose of ibuprofen at suitable dosing intervals to the human patient; performing surgery on the patient to treat the rib fracture; and continuing the intravenous administration of ibuprofen at suitable dosing intervals until the patient no longer requires intravenous dosing of ibuprofen or is discharged from the hospital. In certain preferred embodiments, a suitable therapeutically effective dose of ibuprofen is administered intravenously at suitable dosing intervals for at least 6 hours, for at least about 12 hours, for at least about 24 hours, for at least about 36 hours, or for at least 48 hours following hospital admission, before conversion to oral ibuprofen.

In further embodiments of the above methods, one or more opioid analgesics are administered to the human patient during hospitalization, preferably, in an amount (of opioid analgesic) that is less than that typically required to control pain in human patients (due to the co-administration of intravenous ibuprofen).

In certain preferred embodiments, the human patients receiving 800 mg intravenous ibuprofen as described herein experience a significant reduction in pain as measured, e.g., by the VAS-AUC with movement for the post-operative period (e.g., hours 6-28 after completion of the surgical procedure).

In further embodiments of the above methods, one or more opioid analgesics are administered to the human patient during hospitalization, preferably, in an amount (of opioid analgesic) that is less than that typically required to control pain in human patients (due to the co-administration of intravenous ibuprofen).

In certain preferred embodiments, the human patients receiving the dose of ibuprofen as described herein require the administration of less opioid analgesic (e.g., morphine) than the dose of opioid typically required to provide an equivalent level of pain relief without the administration of intravenous ibuprofen.

In certain preferred embodiments, the human patients receiving the dose of ibuprofen as described herein experience a significant reduction in pain as measured by the VAS at rest area under the curve and by the VRS during hospitalization.

In certain preferred embodiments, the human patients experience less pain via the intravenous administration of ibuprofen as compared to typical patients having a similar injury (e.g., rib fracture) without the benefit of the intravenous administration of ibuprofen.

In preferred embodiments, the human patients receiving the intravenous dose(s) of ibuprofen require significantly less opioid analgesic.

The invention is further directed to a safe and effective method for reducing the amount of opioid analgesic administered to human patients who require hospitalization due to a trauma injury (such as rib fracture), comprising intravenously administering a therapeutically effective (e.g., 800 mg) dose of ibuprofen on the first day of hospitalization (and preferably as soon as possible post-admission to the hospital); and thereafter intravenously administering a therapeutically effective dose of ibuprofen at suitable dosing intervals to the human patient; and continuing the intravenous administration of ibuprofen at suitable dosing intervals until the patient no longer requires intravenous dosing of ibuprofen or is discharged from the hospital. In certain embodiments, the trauma injury/injuries are treated surgically during the hospitalization. In other embodiments, the patient is treated without surgery. In certain preferred embodiments, a suitable therapeutically effective dose of ibuprofen is administered intravenously at suitable dosing intervals for at least 6 hours, for at least about 12 hours, for at least about 24 hours, for at least about 36 hours, or for at least 48 hours following hospital admission, before conversion to oral ibuprofen.

The invention is further directed to method for reducing the length of stay (i.e., length of hospitalization) for human patients who require hospitalization due to a trauma injury (such as rib fracture), comprising intravenously administering an effective (e.g., 800 mg) dose of ibuprofen on the first day of hospitalization (and preferably as soon as possible post-admission to the hospital); and thereafter intravenously administering a therapeutically effective dose of ibuprofen at suitable dosing intervals to the human patient; optionally administering a therapeutically effective dose of an opioid analgesic at suitable dosing intervals; and continuing the intravenous administration of ibuprofen at suitable dosing intervals until the patient no longer requires intravenous dosing of ibuprofen or is discharged from the hospital. In certain embodiments, the trauma injury/injuries are treated surgically during the hospitalization. In other embodiments, the patient is treated without surgery. In certain embodiments, the intravenous administration of ibuprofen in accordance with the method has an opioid-sparing effect on the treatment of the patient. In certain embodiments, the intravenous administration of ibuprofen in accordance with the method reduces the side effects typically associated with opioid analgesic therapy. In certain further embodiments, the intravenous administration of ibuprofen in accordance with the method increases the pain relief attained by the patient, in comparison to patients who have similar injuries who have not received intravenous administration of ibuprofen in accordance with the methods of the present invention.

The invention is further directed to method for reducing pharmacy and/or hospital costs for human patients who require hospitalization due to a trauma injury (such as rib fracture), comprising intravenously administering an effective (e.g., 800 mg) dose of ibuprofen on the first day of hospitalization (and preferably as soon as possible post-admission to the hospital); and thereafter intravenously administering a therapeutically effective dose of ibuprofen at suitable dosing intervals to the human patient; optionally administering a therapeutically effective dose of an opioid analgesic at suitable dosing intervals; and continuing the intravenous administration of ibuprofen at suitable dosing intervals until the patient no longer requires intravenous dosing of ibuprofen or is discharged from the hospital. In certain embodiments, the trauma injury/injuries are treated surgically during the hospitalization. In other embodiments, the patient is treated without surgery. In certain embodiments, the intravenous administration of ibuprofen in accordance with the method has an opioid-sparing effect on the treatment of the patient. In certain embodiments, the intravenous administration of ibuprofen in accordance with the method reduces the side effects typically associated with opioid analgesic therapy. In certain further embodiments, the intravenous administration of ibuprofen in accordance with the method increases the pain relief attained by the patient, in comparison to patients who have similar injuries who have not received intravenous administration of ibuprofen in accordance with the methods of the present invention.

The term “opioid sparing” or “opioid sparing effect” refers to a reduction in opioid analgesic opioid which occurs when the intravenous dose(s) of ibuprofen is combined with a suitable dosing regimen of an opioid analgesic, while providing adequate pain control in the patient requiring hospitalization due to a trauma injury, such as a rib fracture injury. This opioid-sparing effect of the intravenous ibuprofen therapy allows the clinician to diminish the side effects associated with opioid therapy without sacrificing pain control.

### DETAILED DESCRIPTION OF THE INVENTION

NSAIDs are effective adjuncts to opioid analgesia for moderate to severe pain, resulting in pain relief and opioid dose sparing. NSAIDs alone could provide effective analgesia post-surgery when mild to moderate pain is expected. There is also evidence that, by avoiding or decreasing opioid use, NSAIDs can reduce the incidence of opioid associated adverse events.

The present invention is directed to the administration of ibuprofen intravenously to patients who have experienced a trauma injury (such as rib fracture) which requires hospitalization. In accordance with the present invention, a therapeutically effective dose of ibuprofen is administered intravenously preferably on the first day of admission of the patient into the hospital, and preferably, as soon as possible after admission. Thereafter, a suitable therapeutically effective dose of ibuprofen is administered intravenously at suitable dosing intervals following admission, before conversion to oral ibuprofen. In certain embodiments, the therapeutically effective dose of ibuprofen is administered intravenously at suitable dosing intervals for at least 6 hours, for at least about 12 hours, for at least about 24 hours, for at least about 36 hours, or for at least 48 hours following admission.

In certain preferred embodiments of the invention, the patient(s) is being treated for one or more rib fractures, possibly along with other trauma injuries. Examples of trauma injuries which may require hospitalization (and which are therefore encompassed within the appended claims) include, but are not limited to, rib fracture(s), splenic laceration, scalp hematoma, spinal fractures, renal laceration, hip fracture, pubic fracture, pulmonary contusions, soft tissue injury/injuries, adenal hematoma, hepatic contusion, pelvic fracture, humerus fracture, concussion, knee dislocation, laceration(s), forearm fracture, tibia fracture, foot fracture, ankle fracture, combinations of any of the foregoing, and the like.

In preferred embodiments, an opioid analgesic(s) is concomitantly administered to the patient in an amount and dosing interval sufficient to further treat the patient's pain during the patient's hospitalization.

Early intravenous ibuprofen therapy administered in accordance with the present invention significantly decreases opioid analgesic (narcotic) requirement and results in clinically significant decreases in hospital length of stay. Further, the intravenous ibuprofen is well tolerated. In preferred embodiments, intravenous ibuprofen therapy is started upon hospital admission to augment pain control.

As previously stated, the methods of the present invention are directed to the treatment of patients who have experienced a trauma injury which requires hospitalization.

The dose of the intravenous ibuprofen can be from about 400 mg to about 800 mg, every 6 hours. The dosing of intravenous ibuprofen can be administered for, e.g., 48 hours after admission, or can continue further on an every 6 hour or as needed basis, until the patient is discharged from the hospital or is capable of being administered oral pain medications instead.

Intravenous pharmaceutical compositions of ibuprofen include any formulation suitable for administration to a patient via any intravenous method, including a bolus. In some embodiments the rate of infusion is such that the dose is administered over a period of about 30 minutes. In some embodiments the rate of infusion is such that the dose is administered over a period of less than 30 minutes. In some embodiments the rate of infusion is such that the dose is administered over a period of greater than 30 minutes.

In alternative embodiments of the treatment methods described herein, a pharmaceutical formulation comprising ibuprofen is administered to a patient via an injection method. In such embodiments, the pharmaceutical formulation of ibuprofen is a formulation suitable for administration to a patient via the injection method. Suitable injection methods include, in addition to intravenous injection, intraarterial infusion, intramuscular injection, transdermal injection, and subcutaneous injection.

Suitable carriers for intravenous administration include, e.g., physiological saline or phosphate buffered saline (PBS), and solutions containing solubilizing agents, such as glucose, polyethylene glycol, and polypropylene glycol and mixtures thereof.

The formulation may include an aqueous vehicle. Aqueous vehicles include, by way of example and without limitation, Sodium Chloride Injection, Ringers Injection, Isotonic Dextrose Injection, Sterile Water Injection, Dextrose, and Lactated Ringers Injection. Nonaqueous parenteral vehicles include, by way of example and without limitation, fixed oils of vegetable origin, cottonseed oil, corn oil, sesame oil and peanut oil. Antimicrobial agents in bacteriostatic or fungistatic concentrations must be added to parenteral preparations packaged in multiple dose containers which include, by way of example and without limitation, phenols or cresols, mercurials, benzyl alcohol, chlorobutanol, methyl and propyl p hydroxybenzoic acid esters, thimerosal, benzalkonium chloride and benzethonium chloride. Isotonic agents include, by way of example and without limitation, sodium chloride and dextrose. Buffers include, e.g, phosphate and citrate. Antioxidants include, e.g, sodium bisulfate. Local anesthetics include, e.g, procaine hydrochloride. Suspending and dispersing agents include, e.g., sodium carboxymethylcelluose, hydroxypropyl methylcellulose and polyvinylpyrrolidone. Emulsifying agents include, e.g., Polysorbate 80 (TWEEN® 80). A sequestering or chelating agent of metal ions include, e.g., EDTA. Pharmaceutical carriers also include, by way of example and without limitation, ethyl alcohol, polyethylene glycol and propylene glycol for water miscible vehicles and sodium hydroxide, arginine, sodium or potassium phosphate, hydrochloric acid, citric acid, phosphoric acid or lactic acid, among other buffers and excipients for pH adjustment.

Typically a therapeutically effective dosage is formulated to contain a concentration of at least about 0.1% w/w up to about 90% w/w or more, such as more than 1% w/w of ibuprofen.

As used herein a “dosage regimen” refers to the protocol used to administer an intravenous pharmaceutical formulation comprising ibuprofen to a patient. In some embodiments, the dosage regimen comprises a dose amount and dosing interval. In some embodiments the dosage regimen further comprises a dosing duration. As used herein “dosing duration” refers to the period of time over which a dose is administered. For example, if a volume of pharmaceutical composition comprising 400 mg of ibuprofen is administered over a dosing duration of 30 min and administration of a dose is initiated every 6 hours, then the dosage regimen is 400 mg, every six hours, administered over 30 minutes. In some embodiments, the dosage duration is defined simply as 400 mg, every six hours.

In some embodiments described herein, a dosage regimen for post surgical patients is defined as one that results in decreased usage of narcotic analgesic and/or decreased perception of pain and decreased side effects from use of a narcotic analgesic.

All numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

The invention is further directed in part to a method of treating a patient suffering from a trauma injury requiring hospitalization comprising administering to the patient on the first day of hospitalization, and preferably as soon as possible after hospitalization, an intravenous pharmaceutical composition comprising ibuprofen at a dosage of about (i) 400 mg ibuprofen to about (ii) 800 mg ibuprofen. In certain preferred embodiments of this method, the dose of ibuprofen produces a decreased need for narcotic analgesic, decreased side effects from use of a narcotic analgesic and/or decreased perception of pain.

Several prescription and nonprescription brands of ibuprofen are approved for the treatment of fever, pain, and other indications. The recommended over the counter, single dose of oral ibuprofen to treat mild to moderate pain in adults is 400 mg every 4 to 6 hours. For chronic indications, such as rheumatoid arthritis and osteoarthritis, up to 3200 mg/day may be administered (300 mg qid, or 400/600/800 mg tid or qid).

The intravenous ibuprofen formulation of the present invention is the first and only intravenous formulation of ibuprofen available to treat mild to severe pain in adults and to reduce fever in children and adults. Oral ibuprofen has been available for more than 30 years and has a favorable risk/benefit profile. The intravenous formulation is now commercially available in U.S. in the 4-mL and 8-mL vials (each 100 mg/mL) for dilution in either saline or dextrose solution.

Examples of suitable IV ibuprofen treatments in accordance with the invention include the following: for simple adult dosing for pain: 800 mg about every 6 hours, although certain patients may require higher doses for pain management. In such patients, the dose may be adjusted up to 800 mg, not to exceed 3200 mg total daily dose.

As described herein, in a retrospective review it was determined that intravenous administration of ibuprofen to human patients who require hospitalization due to a trauma injury (such as rib fracture) on the first day of hospitalization (and preferably as soon as possible post-admission to the hospital) and thereafter for at least about 48 hours post-admission significantly decreases narcotic requirement and results in clinically significant decreases in hospital length of stay, without complications related to ibuprofen administration.

The use of intravenous ibuprofen formulations in accordance with the present invention provide, e.g., the following benefits: speed relief of pain and expedite release from a hospital or hospital-like setting; and an opioid-sparing effect, thereby reducing the narcotic requirements. It may also provide a reduction in pain at rest and with movement as measured by visual analog scores (VAS); a reduction in opioid side effects (nausea, vomiting, constipation); and may provide an improvement in time to ambulation may enable facilities to schedule additional procedures in the ambulatory setting.

In preferred embodiments, the intravenous ibuprofen provides an opioid-sparing effect, enabling the reduction of the dose of opioid to the patients. Significant daily narcotic requirement reductions were seen, particularly on days 3 through 7 after hospitalization. However, many patients in the retrospective review did not receive intravenous administration of ibuprofen on the first day. It is believed that significant daily narcotic requirement reductions will be seen on day 2 and preferably on day 1, as well when intravenous administration of ibuprofen is begun as early after hospital admission as possible. Preferably, the human patients receiving intravenous ibuprofen experience at least a 10% reduction in mean opioid consumption on at least one day after hospitalization. In certain preferred embodiments, the human patient(s) receiving intravenous ibuprofen have about a 16% reduction, or about a 20% reduction, or at least a 25% reduction, or at least a 30% reduction, or at least a 40% reduction in mean opioid (e.g., morphine) consumption on at least one day after hospitalization, and in certain preferred embodiments, on days 3 through the end of hospitalization (e.g., day 7).

The opioids are a group of drugs, both natural and synthetic, that are employed primarily as centrally-acting analgesics and are opium or morphine-like in their properties. The opioids include morphine and morphine-like homologs, including, e.g., the semisynthetic derivatives codeine (methylmorphine) and hydrocodone (dihydrocodeinone) among many other such derivatives. Morphine and related opioids exhibit agonist activity at central nervous system or CNS (referring to the brain and spinal cord) mu opioid receptors as well as showing affinity for the delta and kappa opioid receptors, to produce a range of effects including analgesia, drowsiness, changes in mood and mental clouding. In addition to potent analgesic effects, the morphine-related opioids may also cause a number of undesirable effects, including, for example, respiratory depression, nausea, vomiting, dizziness, mental clouding, dysphoria, pruritus, constipation, increased biliary tract pressure, urinary retention and hypotension.

Although the data in Example 1 below is expressed in terms of a dose of morphine or an equivalent dose of morphine, one skilled in the art will recognize that other opioid analgesics can be used instead of part or all of the morphine. Opioid analgesics which may be used in accordance with the invention include, but are not limited to, alfentanil, allylprodine, alphaprodine, anileridine, benzylmorphine, bezitramide, buprenorphine, butorphanol, clonitazene, cyclazocine, desomorphine, dextromoramide, dezocine, diampromide, diamorphone, dihydrocodeine, dihydromorphine, dimenoxadol, dimepheptanol, dimethylthiambutene, dioxaphetylbutyrate, dipipanone, eptazocine, ethoheptazine, ethylmethylthiambutene, ethylmorphine, etonitazene, fentanyl, heroin, hydromorphone, hydroxypethidine, isomethadone, ketobemidone, levallorphan, levorphanol, levophen-acylmorphan, lofentanil, meperidine, meptazinol, metazocine, methadone, metopon, morphine, myrophine, nalbuphine, narceine, nicomorphine, norlevorphanol, normethadone, nalorphine, normorphine, norpipanone, opium, oxycodone, oxymorphone, papavereturn, pentazocine, phenadoxone, phenomorphan, phenazocine, phenoperidine, piminodine, piritramide, propheptazine, promedol, properidine, propiram, propoxyphene, sufentanil, tilidine, tramadol, salts thereof, complexes thereof; mixtures of any of the foregoing, mixed mu-agonists/antagonists, mu-antagonist combinations salts or complexes thereof, and the like. In certain preferred embodiments, the opioid analgesic is a mu or kappa opioid agonist. In certain preferred embodiments, the opioid analgesic is morphine, dihydrocodeine, hydromorphone, fentanyl, oxycodone, oxymorphone, salts thereof, and mixtures of any of the foregoing.

### DETAILED DESCRIPTION OF PREFERRED EMBODIMENTS

The following example represents specific embodiments of the foregoing discovery, and is not representative of the entire scope of the invention.

### Example 1

A retrospective review of traumatic rib fracture patients managed according to a predefined guideline was performed at a Level 1 trauma center (Orlando Regional Trauma Center). Unless contraindicated, patients received intravenous ibuprofen for at least 48 hours following admission before conversion to oral ibuprofen. Patients who received intravenous ibuprofen and narcotics for pain control were matched by age and number of rib fractures to patients who received narcotics alone (control group). Pain medication requirement was evaluated for the first seven days of hospitalization. All medication dosages were converted to either IV morphine or intravenous ibuprofen to facilitate comparison. Complications related to ibuprofen administration were collected. Data are reported as mean±standard deviation and compared using Mann-Whitney U-test.

Chart data from 21 admitted rib fracture patients who received intravenous ibuprofen (Caldolor®, commercially available from Cumberland Pharma, Inc.) in their analgesic regimen was collected. As a control, 21 matched historical control rib fracture patients who had received no ibuprofen therapy were identified. Information concerning the age of the patients in the review and their injuries, divided between patients receiving intravenous ibuprofen (the “Caldolor” group) and the control group is provided in Table 1.

TABLE 1
Patient Information

| | Treatment Group | Control Group |
|---|---|---|
| | N = 21 | N = 21 |
| Age (yrs): | 52.05 (SD 14.19) | 53.38 (SD 15.52) |
| # Rib Fx*: | 4.86 (SD 2.78) | 4.24 (SD 1.81) |

*Fx means “fracture”

Narcotic usage was reviewed and all drugs were converted to an equivalent IV-morphine dose using the conversions provided in Table 2 below:

TABLE 2
Narcotic (Opioid) Analgesics converted to Morphine IV:

| | | | MG PO | MG IV |
|---|---|---|---|---|
| | Base Opiate | | Morphine | Morphine |
| Oxycodone/APAP | 20 | mg | 30 mg | 10 mg |
| (Percocet) | oxycodone | | | |
| Oxycontin | 20 | mg | 30 mg | 10 mg |
| | oxycodone | | | |
| OxyIR (oxycodone) | 20 | mg | 30 mg | 10 mg |
| | oxycodone | | | |
| Hydromorphone (Dilauded) | 1.5 | mg | — | 10 mg |
| IV | | | | |
| Hydromorphone (Dilauded) | 7.5 | mg | 30 mg | 10 mg |
| PO | | | | |
| Fentanyl | 100 | mcg | — | 10 mg |

NSAIDs converted to IV Ibuprofen

| | Dose | IV Ibuprofen | Oral Ibuprofen |
|---|---|---|---|
| Ibuprofen | — | 800 mg | 680 mg |

The mean daily dose of intravenous ibuprofen was 1070 mg±880 mg. The mean daily intravenous narcotic dose was 18.9 mg±15.6 mg for the Treatment group versus 32.1 mg±24.0 mg for the Control group (p<0.0001).

The daily narcotic requirement did not differ between the Treatment Group and the Control Group on Day 1 or Day 2. However, narcotic requirement was significantly decreased in the Treatment Group on each of Days 3 through 7 (p<0.05). There were no significant complications associated with ibuprofen therapy. The results demonstrate that those patients who had ibuprofen (IV and oral) integrated into their analgesic regimen used less narcotics than those who did not receive any ibuprofen.

Looking at the data more closely, Table 3 provides a comparison in the drug use in the Treatment group (narcotic usage; intravenous ibuprofen (“Caldolor”) usage and oral ibuprofen (“PO IBU”) usage) and drug use in the “Matched” Control group (narcotic usage):

TABLE 3

| | Caldolor Exposure | | | | | | | | | Matched Control | | |
|---|---|---|---|---|---|---|---|---|---|---|---|---|
| | Narcotic Usage | | | IV IB Usage | | | PO IBU Usage | | | Narcotic Usage | | |
| | N | mg morphine | SD | N | mg | SD | N | mg | SD | N | mg morphine | SD |
| Day 1 | 21 | 23.52 | 18.15 | 21 | 1447.62 | 695.43 | 1 | 800.00 | — | 21 | 16.93 | 14.89 |
| Day 2 | 20 | 23.53 | 15.90 | 20 | 1960.00 | 869.60 | 8 | 1250.00 | 498.57 | 21 | 34.94 | 20.95 |
| Day 3 | 16 | 19.95 | 14.64 | 11 | 1927.27 | 993.07 | 9 | 2133.33 | 979.80 | 20 | 36.48 | 25.74 |
| Day 4 | 11 | 15.45 | 14.63 | 5 | 1600.00 | 1131.37 | 9 | 1955.56 | 988.83 | 17 | 36.65 | 25.43 |
| Day 5 | 8 | 10.73 | 12.39 | 2 | 2000.00 | 1697.06 | 6 | 2800.00 | 669.33 | 14 | 39.95 | 27.68 |
| Day 6 | 7 | 11.07 | 9.56 | 1 | 1600.00 | — | 6 | 2533.33 | 1063.33 | 11 | 33.30 | 29.43 |
| Day 7 | 5 | 9.00 | 8.22 | 0 | 0.00 | — | 3 | 3200.00 | 0.00 | 7 | 28.43 | 17.96 |

The data collected also suggests that patients who received Caldolor (and oral ibuprofen) as part of their analgesic regimen had a reduced length of hospital stay—almost a day difference (based on mean data). Length of hospital stay (mean data) were 4.42 days±3.35 days for the Treatment group versus 5.41 days±2.89 days for the Control Group (p=0.17). Length of hospital stay (median data) were 3.24 days for the Treatment group versus 7.48 days for the Control Group.

In Table 3, in the far right (Matched Controls) group, it is significant to note that after day 1, morphine usage is approximately 35 mg per day. In contrast, in the Caldolor exposure (Treatment) group, it can be seen that for patients receiving ibuprofen in their analgesic regimen, morphine usage decreases to approximately 20 mg per day after day 1. It is important to note that not everyone in the Treatment group actually received ibuprofen every day. Few to none in the Treatment group received 3200 mg (recommended max daily dose) ibuprofen in their multi-modal therapy. Further, it is important to note that not every patient received ibuprofen early in their hospitalization—some patients did not receive ibuprofen until days 3, 4 or 5 of their hospital stay. Despite the sub-optimal dosing, there was still a substantial narcotic sparing effect observed.

Table 4 provides a summary of the percent reduction in opioid administration to the Treatment group as compared to the control group. As can be seen from Table 4, there was a greater than 32% reduction in opioid usage in the Treatment group for day 2, greater than 45% reduction in opioid usage in the Treatment group for day 3, greater than 57% reduction in opioid usage in the Treatment group for day 4, greater than 73% reduction in opioid usage in the Treatment group for day 5, greater than 66% reduction in opioid usage in the Treatment group for day 6, and greater than 68% reduction in opioid usage in the Treatment group for day 7.

TABLE 4

| | Caldolor | | | | | | | |
|---|---|---|---|---|---|---|---|---|
| | Exposure | | | | | | | |
| | | mg | | Matched Control | | | | |
| | | mor- | | | mg | | p- | % |
| | N | phine | SD | N | morphine | SD | value* | reduction |
| Day 1 | 21 | 23.52 | 18.15 | 21 | 16.93 | 14.89 | 0.32 | −38.92% |
| Day 2 | 20 | 23.53 | 15.90 | 21 | 34.94 | 20.95 | 0.12 | 32.66% |
| Day 3 | 16 | 19.95 | 14.64 | 20 | 36.48 | 25.74 | 0.025 | 45.31% |
| Day 4 | 11 | 15.45 | 14.63 | 17 | 36.65 | 25.43 | 0.001 | 57.84% |
| Day 5 | 8 | 10.73 | 12.39 | 14 | 39.95 | 27.68 | 0.006 | 73.14% |
| Day 6 | 7 | 11.07 | 9.56 | 11 | 33.30 | 29.43 | 0.024 | 66.76% |
| Day 7 | 5 | 9.00 | 8.22 | 7 | 28.43 | 17.96 | 0.051 | 68.34% |

*Mann-Whitney U

Additionally, data was collected with regard to pharmacy and hospital costs for patients who were treated with intravenous ibuprofen early (e.g., day 1) in their hospitalization (“Early Treatment group”) versus patients who received intravenous ibuprofen later during their hospitalization (i.e., after Day 2, referred to herein as the “Late Treatment group”).

The data collected suggests that patients who received Early Treatment group as a part of their analgesic regimen had reduced pharmacy and hospital costs ($). The pharmacy costs (mean data) were $4,550 (SD $4,018) for the Early Treatment group versus $11,719 (SD $6,396) for the Late Treatment group. The hospital costs (mean data) were $38,671 (SD $28,109) for the Early Treatment group versus $95,981 (SD $68,201) for the Late Treatment group.

### CONCLUSIONS

The results indicate that treatment of trauma patients who require hospitalization with intravenous ibuprofen is a safe and effective analgesic option for the management of pain, and which results in a shortened length of stay, a reduction in concomitantly administered opioid analgesics, and reduced pharmacy and hospital costs. It is further believed that the methods of the present invention result in a decrease of side effects normally associated with treatment of such trauma injuries and increased pain relief.

It will be readily apparent to one of ordinary skill in the relevant arts that other suitable modifications and adaptations to the methods and applications described herein are suitable and may be made without departing from the scope of the invention or any embodiment thereof. While the invention has been described in connection with certain embodiments, it is not intended to limit the invention to the particular forms set forth, but on the contrary, it is intended to cover such alternatives, modifications and equivalents as may be included within the spirit and scope of the invention as defined by the following claims.

1. A method for reducing the length of hospital stay for human patients who require hospitalization due to a trauma injury, comprising intravenously administering a first dose of a therapeutically effective dose of ibuprofen as soon as possible after hospitalization of patients suffering from a trauma injury; and thereafter intravenously administering a therapeutically effective dose of ibuprofen at suitable dosing intervals to the human patients until intravenous dosing of ibuprofen is no longer required.

2. The method of claim 1, wherein the therapeutically effective dose of ibuprofen at suitable dosing intervals to the human patients until at least about 48 hours after administration of the first dose of ibuprofen.

3. The method of claim 1, wherein the therapeutically effective dose of ibuprofen at suitable dosing intervals to the human patients until the patients are discharged from the hospital.

4. The method of claim 1, further comprising administering to the human patient a therapeutically effective dose of an opioid analgesic on the first day of hospitalization, and thereafter administering a therapeutically effective dose of an opioid analgesic in suitable dosing intervals to the human patient until the patient no longer requires treatment with an opioid analgesic, or is discharged from hospitalization.

5. The method of claim 1, wherein the trauma injury is one or more rib fractures.

6. The method of claim 1, further comprising intravenously administering a dose of ibuprofen as soon as possible after hospitalization, and thereafter administering a therapeutically effective dose of ibuprofen every six hours for about 48 hours or more.

7. The method of claim 6, wherein the intravenous dose of ibuprofen is about 800 mg.

8. The method of claim 5, further comprising administering a maximum daily dose of about 3200 ibuprofen to the patient during hospitalization.

9. The method of claim 4, further comprising intravenously administering the intravenous ibuprofen to patients in a sufficient dose to provide an opioid-sparing effect, enabling the reduction of the dose of opioid to the patients.

10. The method of claim 2, wherein the dose of opioid analgesic is reduced at least about 16% over a period of a day during the hospitalization of the patient.

11. The method of claim 2, wherein the dose of opioid analgesic is reduced at least about 30% over a period of a day during the hospitalization of the patient.

12. The method of claim 4, further comprising administering the intravenous ibuprofen in a sufficient dose to provide a reduction in side effects associated with the administration of opioid analgesics.

13. The method of claim 1, further comprising intravenously administering the ibuprofen in a sufficient dose such that the patients have a shortened length of stay in the hospital than if the intravenous ibuprofen is not administered.

14. The method of claim 4, further comprising intravenously administering the ibuprofen in a sufficient dose such that the patients have a shortened length of stay in the hospital than if the intravenous ibuprofen is not administered.

15. The method of claim 1, wherein the first dose of intravenous ibuprofen is administered as soon as possible on the first day of hospitalization.

16. The method of claim 4, further comprising administering the intravenous ibuprofen in a sufficient dose to reduce pain scores in patients who are concurrently administered opioid analgesics, as compared to similar patients receiving opioid analgesics without intravenous ibuprofen.

17. A method for treating human patient who requires hospitalization for a trauma injury, comprising intravenously administering a first dose of a therapeutically effective dose of ibuprofen intravenously as soon as possible after hospitalization of a patient suffering from a trauma injury; thereafter intravenously administering a therapeutically effective dose of ibuprofen at suitable dosing intervals to the human patient until the patient no longer requires intravenous dosing of ibuprofen; and concomitantly administering an effective dose of an opioid analgesic to the patient at suitable dosing intervals.

18. The method of claim 17, further comprising intravenously administering the ibuprofen in a sufficient dose such that the patients have a shortened length of stay in the hospital than if the intravenous ibuprofen is not administered.

19. The method of claim 17, wherein the trauma injury is rib fracture.

20. The method of claim 17, wherein the intravenous dose of ibuprofen is about 800 mg.

21. The method of claim 17, further comprising administering a maximum daily dose of about 3200 ibuprofen to the patient during hospitalization.

22. The method of claim 17, wherein the dose of opioid analgesic is reduced at least about 16% over a period of a day during the hospitalization of the patient, as compared to similar patients receiving opioid analgesics without intravenous ibuprofen.

23. The method of claim 17, wherein the dose of opioid analgesic is reduced at least about 30% over a period of a day during the hospitalization of the patient, as compared to similar patients receiving opioid analgesics without intravenous ibuprofen.

24. The method of claim 17, wherein a suitable therapeutically effective dose of ibuprofen is administered intravenously at suitable dosing intervals for about 48 hours following hospital admission, before conversion of ibuprofen treatment to oral ibuprofen.

25. A method for reducing the length of hospital stay for human patients who require hospitalization due to a trauma injury (such as rib fracture), comprising intravenously administering a first dose of a therapeutically effective dose of ibuprofen as soon as possible after hospitalization of patients suffering from a trauma injury; thereafter intravenously administering a therapeutically effective dose of ibuprofen at suitable dosing intervals to the human patients until the patient no longer requires intravenous dosing of ibuprofen; and concomitantly administering an effective dose of an opioid analgesic to the patients at suitable dosing intervals.

26. The method of claim 25, further comprising converting the patients from intravenous ibuprofen treatment to oral ibuprofen treatment at or before discharge of the patient from the hospital.

27. The method of claim 25, wherein the trauma injury comprises rib fracture.

28. The method of claim 25, wherein the intravenous dose of ibuprofen is about 800 mg.

29. The method of claim 25, further comprising administering a maximum daily dose of about 3200 ibuprofen to the patient during hospitalization.

30. The method of claim 25, wherein the dose of opioid analgesic is reduced at least about 16% over a period of a day during the hospitalization of the patient, as compared to similar patients receiving opioid analgesics without intravenous ibuprofen.

31. The method of claim 25, wherein the dose of opioid analgesic is reduced at least about 30% over a period of a day during the hospitalization of the patient, as compared to similar patients receiving opioid analgesics without intravenous ibuprofen.

32. The method of claim 25, wherein the mean length of stay of the patients is about 1 day less than similar patients receiving opioid analgesics without intravenous ibuprofen.

33. The method of claim 25, further comprising intravenously administering a therapeutically effective dose of ibuprofen at suitable dosing intervals to the human patients for at least about 48 hours after administration of the first dose of ibuprofen.
